(19) United States
(12) Patent Application Publication
(10) Pub. No.: US 2007/0255308 A1
(43) Pub. Date: Nov. 1, 2007

(54) Procedural cannula for transgastric surgical procedures

(76) Inventors: Michael S. Williams, Santa Rosa, CA (US); William L. Athas, Chapel Hill, NC (US); Jeffrey A. Smith, Petaluma, CA (US); Geoffrey A. Orth, Sebastopol, CA (US); Richard A. Glenn, Chapel Hill, CA (US)

Correspondence Address: STALLMAN & POLLOCK LLP, 353 SACRAMENTO STREET, SUITE 2200, SAN FRANCISCO, CA, 94111, US

(21) Appl. No.: 11/655,445

(22) Filed: Jan. 19, 2007

Related U.S. Application Data
(63) Continuation-in-part of application No. 11/528,009, filed on Sep. 27, 2006.
(60) Provisional application No. 60/720,943, filed on Sep. 27, 2005.
(60) Provisional application No. 60/794,563, filed on Apr. 24, 2006.
(60) Provisional application No. 60/826,535, filed on Sep. 21, 2006.
(60) Provisional application No. 60/760,132, filed on Jan. 19, 2006.

(51) Int. Cl. A61M 29/00

(52) U.S. Cl. 606/198

(57) ABSTRACT

In a system and method for performing a treatment within a body cavity, an elongate cannula is provided having one or more distally positioned expandable elements. An incision is formed in body tissue to gain access to a body cavity. A distal portion of the cannula is extended through the opening and into the body cavity. The expandable element is expanded within the body cavity. During expansion, a portion of the expandable contacts tissue or structures within the body cavity, such that the expansion causes the distal portion of the cannula to deflect generally away from the tissue or structures.

### RELATED APPLICATIONS

This application claims the benefit of U.S. Provisional Application No. 60/760,132, filed Jan. 19, 2006, and is a continuation in part of U.S. application Ser. No. 11/528,009, filed Sep. 27, 2006, which in turn claims the benefit of U.S. Provisional Application Nos. 60/720,943, filed Sep. 27, 2005, 60/794,563, filed Apr. 24, 2006, and 60/826,535, filed Sep. 21, 2006.

### FIELD OF THE INVENTION

The present invention relates to the field of devices and procedures for use in performing surgery in the peritoneal cavity.

### BACKGROUND OF THE INVENTION

Surgery in the abdominal cavity is typically performed using open surgical techniques or laparoscopic procedures. Each of these procedures requires incisions through the skin and underlying muscle and peritoneal tissue, and thus results in the potential for post-surgical scarring and/or hernias.

Systems and techniques in which access to the abdominal cavity is gained through the esophagus, stomach and/or intestine are advantageous in that incisions through the skin and underlying muscle and peritoneal tissue may be avoided.

### BRIEF DESCRIPTION OF THE DRAWINGS

FIG. 1 is a schematic drawings illustrating use of an access cannula to gain access through the stomach to the abdominal cavity

FIG. 2 is similar to FIG. 1 and additionally shows introduction of a procedural cannula through the access cannula into the abdominal cavity.

FIG. 3 is similar to FIG. 2 and further shows deflection of the procedural cannula due to the expansion of its expandable devices into contact with surrounding tissue. The figure additionally shows instruments passing through the procedural cannula for use in performing a treatment within the abdominal cavity.

FIG. 4 is a perspective view of a procedural cannula showing the expandable devices prior to expansion.

FIGS. 5-8 are perspective views of procedural cannulas having various arrangements of expandable devices. The expandable devices are shown in the expanded position.

### DETAILED DESCRIPTION OF THE DRAWINGS

Applicant's prior application Ser. No. 11/528,009, SURGICAL DEVICES AND PROCEDURES, filed Sep. 27, 2006 TRANSGASTRIC describes an embodiment of a surgical access cannula for use in gaining access to the peritoneal cavity of a patient. The cannula is configured such that its distal end may be advanced orally through the esophagus and into the peritoneum—and preferably into the stomach or intestine. Once the access cannula is positioned in the stomach or intestine, an instrument (e.g. a scalpel, needle, or equivalent device) is passed through the access cannula are used to form an incision in the stomach or intestinal wall giving access to the peritoneal cavity. Elements of the access cannula create sealed access through the incision, permitting passage of instruments into the peritoneal cavity via a re-sealable valve.

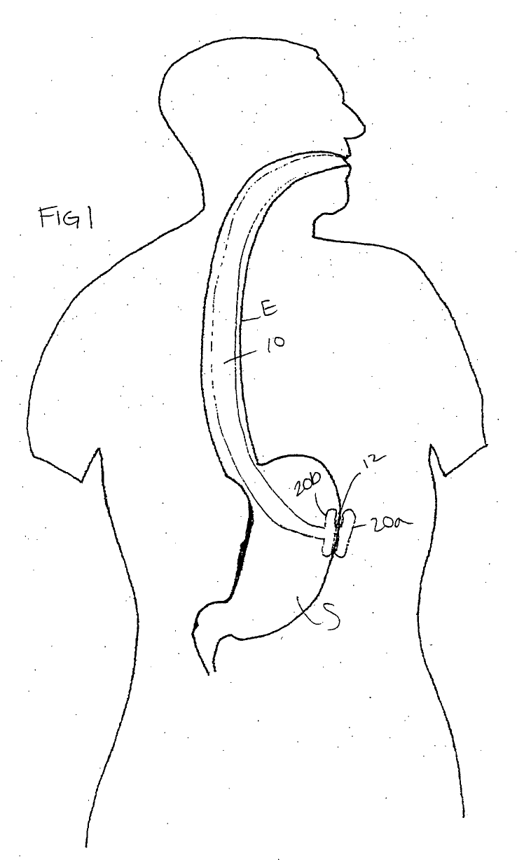

FIG. 1 generally illustrates use of one embodiment of the type described in Applicant's prior application Ser. No. 11/528,009. In this example, access to the peritoneal cavity is gained through the stomach although many other uses for the system are conceivable (e.g. access to the peritoneal cavity via vaginal insertion of the procedural cannula and formation of an incision in the vagina or uterus).

Use of the illustrated system includes passing the distal end 12 of an access cannula 10 into the mouth of a patient and extending it through the esophagus E into the stomach S (or, in alternative embodiments, the intestine). An incision or perforation is formed in the wall of the stomach using an instrument passed through the cannula 10 or inserted separately into the stomach as discussed in greater detail in the prior application. The distal end 12 of the cannula 10 is passed through the perforation P, such that a distal sealing member 20a on the cannula is positioned outside of the stomach and a proximal most sealing member 20b on the cannula remains inside the stomach. Inflation fluid is delivered to inflate the distal sealing member 20a. Once the distal sealing member 20a has been inflated, traction is applied to the cannula 10 to draw the distal sealing member 20a into firm contact with the stomach wall. Inflation fluid is then delivered to inflate the proximal sealing member 20b, causing the stomach wall to be engaged between the sealing members 20a, 20b, and further causing the sealing members 20a, 20b to seal the perforation P against passage of fluids and/or gases.

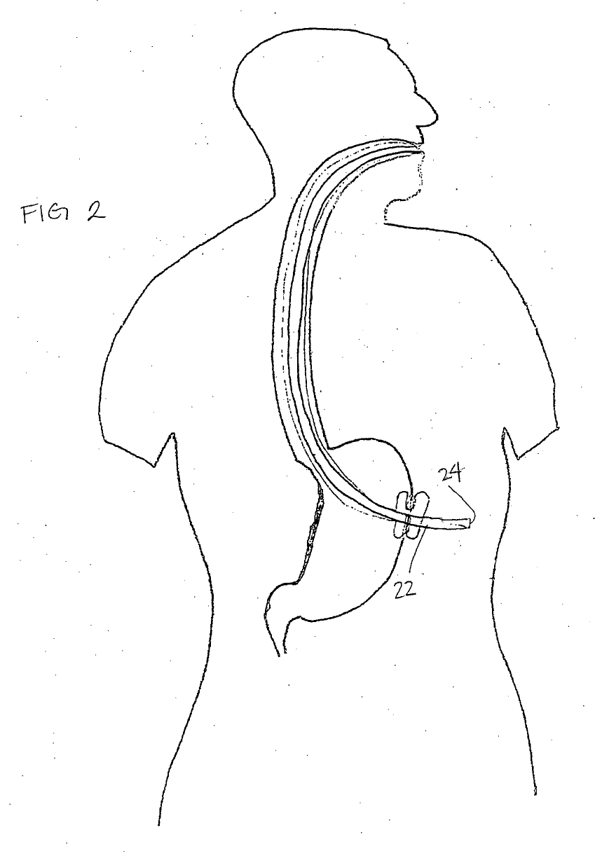

Referring to FIG. 2, a procedural cannula 22 includes an opening 24 at or near its distal end. During use, one or more procedural cannulas 22 are inserted into the cannula 10. Each procedural cannula 22 is advanced such that the opening 24 is disposed within the peritoneal cavity as shown in FIG. 2. Instruments (not shown in FIG. 2) to be used in carrying out the procedure are then advanced through the procedural cannula 22 into the peritoneal cavity.

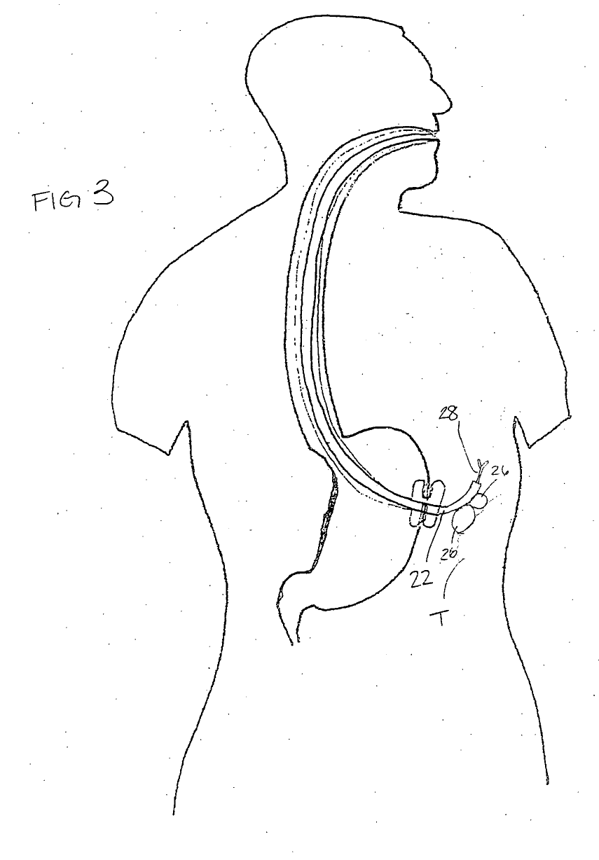

The procedural cannula 22 preferably includes features that allow its distal opening 24 to be oriented generally towards the target position for the instruments that are to gain access into the peritoneal cavity via the cannula 22. Referring to FIG. 3, in one embodiment the procedural cannula 22 is provided with one or more expandable elements 26 that are expanded within the peritoneal cavity. The expandable elements may be inflatable balloons, mechanically expandable frameworks, or other structures capable of being passed into the body in a compressed state and then later expanded. Expansion is achieved using features positioned at the proximal end of the procedural cannula, outside the body. Such features might include, for example, sources of inflation gas fluidly coupled to distal expansion balloons of the type shown in the figures, pullwires manipulatable from outside the body, or a sheath compressing an expansion element and retractable to allow expansion of a mechanical expansion element.

During expansion, the elements 26 expand into contact with body tissue T within the peritoneal cavity, thus causing deflection of the cannula as shown in FIG. 3. The positioning and orientation of the expandable elements may be selected based upon the location of the incision in the stomach relative to the target location (e.g. the gallbladder) for instruments 28 to be delivered through the cannula 22.

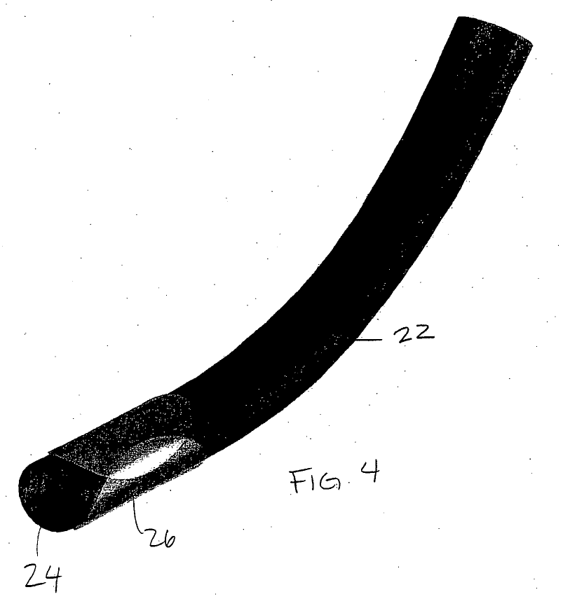

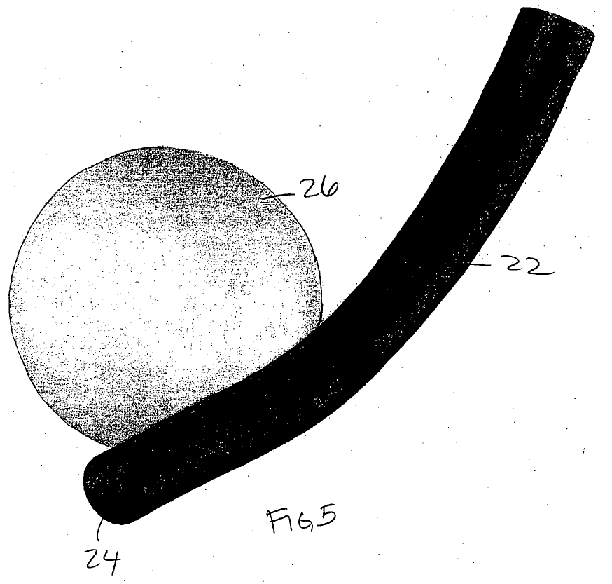

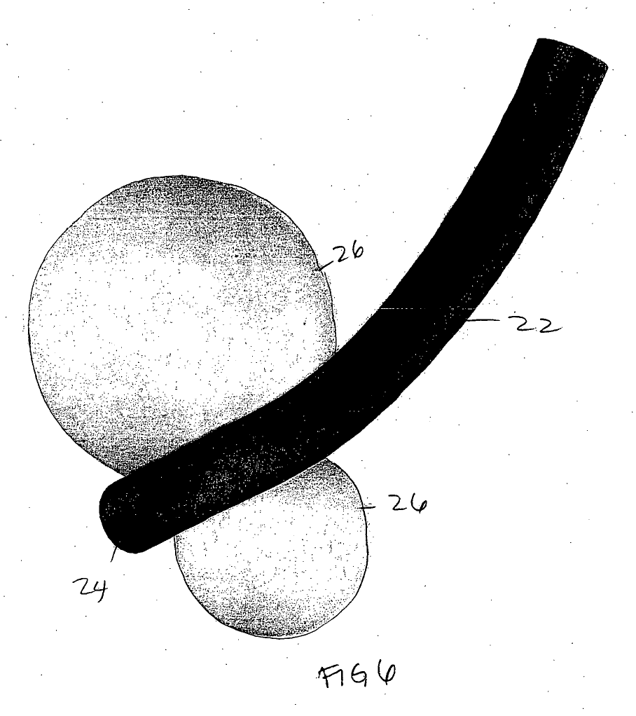

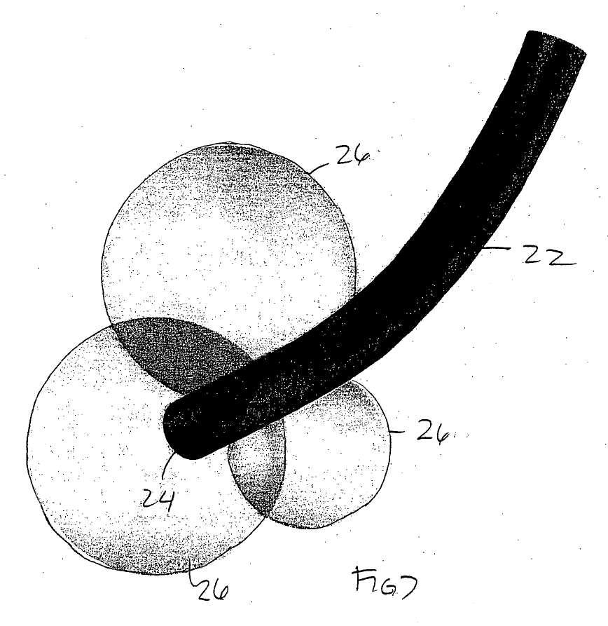

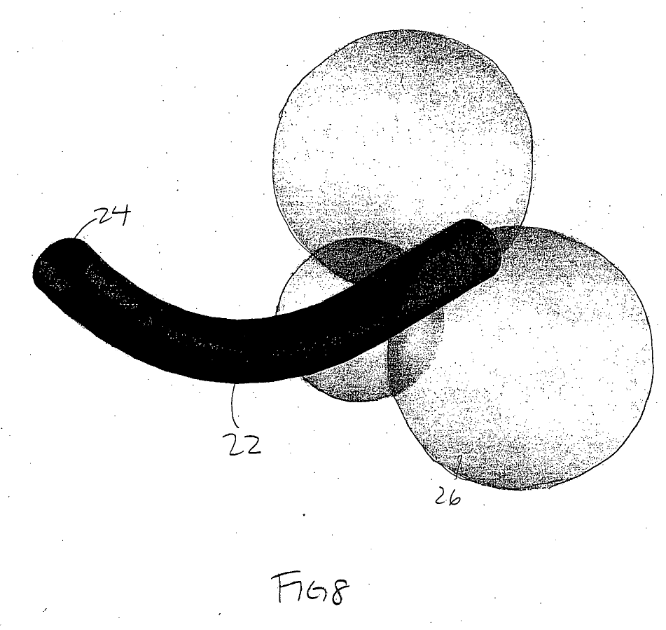

FIG. 4 illustrates the cannula 22 with expandable elements 26 in the unexpanded state. FIGS. 5-8 illustrate four examples of suitable arrangements of expandable elements. The FIG. 5 embodiment utilizes a single element 26, thus providing deflection of the cannula in a single direction. Embodiments such as the FIG. 5-8 embodiments include multiple elements 26. These elements may be simultaneously expanded and collapsed, or they may be expanded and collapsed independently of one another to allow repositioning and/or re-orientation of the distal opening 24 as needed during the course of a procedure.

Alternative means for deflecting the cannula 22 include elements such as pull wires, shaped mandrels, or other features that can be used to bend or deflect the cannula 22 as needed.

While certain embodiments have been described above, it should be understood that these embodiments are presented by way of example, and not limitation. It will be apparent to persons skilled in the relevant art that various changes in form and detail may be made therein without departing from the spirit and scope of the invention. This is especially true in light of technology and terms within the relevant art(s) that may be later developed.

Any and all patents, patent applications and printed publications referred to above are incorporated by reference.

We claim:

1. A method of treating body tissue, comprising the steps of:
providing an elongate first cannula having at least one distally positioned expandable element;
forming an opening in a tissue wall;
passing a distal portion of the first cannula through the opening and into a body cavity, the first cannula assuming a first orientation within the body cavity;
expanding the expandable element within the body cavity such that the expandable element expands against a body structure within the body cavity, causing the distal portion of the first cannula to deflect to a second orientation within the body cavity.

2. The method of claim 1, wherein passing the method includes introducing the first cannula through a natural body orifice prior to passing the distal portion through the opening.

3. The method of claim 2, wherein the natural orifice is selected from the group of natural body orifices consisting of a mouth, a rectum and a vagina.

4. The method of claim 1, wherein the method further includes positioning a second cannula in the opening, and wherein passing the distal portion of the first cannula through the opening includes passing the distal portion of the first cannula through the second cannula.

5. The method of claim 4, wherein forming the opening includes introducing the second cannula through a natural body orifice, passing an instrument through the second cannula and using the instrument to form the opening, and inserting the second cannula into the opening after forming the opening.

6. The method of claim 4, wherein the method includes anchoring a portion of the second cannula in the opening.

7. The method of claim 6, wherein anchoring a portion of the second cannula includes expanding a first anchor distally of the opening.

8. The method of claim 7, wherein anchoring a portion of the second cannula includes expanding a second anchor proximally of the opening.

9. The method of claim 1, wherein the method includes passing an instrument through the first cannula and using the instrument to perform a treatment in the body cavity.

10. The method of claim 1, wherein the method includes expanding a second expandable element on the distal portion of the first cannula, said expansion of the second element causing the first cannula to deflect to a third orientation within the body cavity, the third orientation being different from the second orientation.

11. The method of claim 10, where the method includes retracting the second element to allow the first cannula to deflect to the third orientation.

12. The method of claim 1, wherein expanding the expandable element includes introducing inflation medium into the expandable element.

13. A procedural cannula comprising:
a cannula proportioned for insertion into a body cavity, the cannula including a first expandable element at the distal portion of the cannula, the cannula sufficiently flexible such that expansion of the first expandable element into contact with a body structure within the body cavity causes deflection of the distal portion within the body cavity.

14. The procedural cannula of claim 13, further including a second expandable elements, the first and second expandable elements independently expandable such that expansion of the first expandable element against tissue within a body cavity causes the cannula to deflect to a first position, and expansion of the second element against a body structure within the body cavity causes the procedural cannula to deflect to a second position different than the first position.

15. A cannula system for use in medical procedures, the cannula system including:
an access cannula, the access cannula including an anchor engageable with an opening formed in body tissue between a first body cavity and a second body cavity; and
a procedural cannula extendable through the access cannula, the procedural cannula including a first expandable element at the distal portion of the procedural cannula, the procedural cannula sufficiently flexible such that expansion of the first expandable element into contact with a body structure within the second body cavity causes deflection of the distal portion within the second body cavity.

16. The cannula system of claim 15, further including an instrument extendable through the access cannula, the instrument operable to form the opening in the body tissue.

17. The cannula system of claim 15, further including an instrument extendable through the procedural cannula, the instrument operable to perform a treatment in the body cavity.

18. The cannula system of claim 15, wherein the first expandable element is an inflatable balloon.

19. The cannula system of claim 15, further including a second expandable elements, the first and second expandable elements independently expandable such that expansion of the first expandable element against tissue within a body cavity causes the cannula to deflect to a first position, and expansion of the second element against a body structure within the body cavity causes the procedural cannula to deflect to a second position different than the first position.

20. The cannula system of claim 15, wherein the access cannula is proportioned to extend through a mouth to a stomach wall, and wherein the access cannula is proportioned to extend through the mouth and through the stomach will to a peritoneal cavity.
